(19) United States
(12) Patent Application Publication
(10) Pub. No.: US 2025/0046449 A1
(43) Pub. Date: Feb. 6, 2025

(54) SYSTEMS AND METHODS FOR INTELLIGENT IDENTIFICATION OF FALL EVENTS

(71) Applicant: Optum, Inc., Minnetonka, MN (US)

(72) Inventors: Anurag SINHA, Noida (IN); Arun Kumar TIWARI, New Delhi (IN); Amardeep SHARMA, Delhi (IN); Anirban SENGUPTA, Gurugram (IN); Lisa A. SCHMIDT, San Antonio, TX (US); Derick W. YOUNG, San Antonio, TX (US)

(21) Appl. No.: 18/365,461

(22) Filed: Aug. 4, 2023

(51) Int. Cl. G16H 50/20; G06F 40/284; G16H 10/60; G16H 70/60

(52) U.S. Cl. CPC G16H 50/20; G06F 40/284; G16H 10/60; G16H 70/60

(57) ABSTRACT

A method performed by one or more processors includes: receiving at least one first data object and at least one second data object for an entity; determining if the at least one first data object includes at least one inclusionary code associated with a fall related injury; determining if the at least one first data object excludes at least one exclusionary code associated with a non-fall related injury; and when it is determined that the at least one first data object includes the at least one inclusionary code and excludes the at least one exclusionary code: determining if the at least one second data object includes at least one fall-related word; and when it is determined that the at least one second data object includes the at least one fall-related word, marking the at least one first data object as being indicative of an actual fall event.

### TECHNICAL FIELD

Various embodiments of the present disclosure relate generally to systems and methods for healthcare data analytics, and more particularly to systems and methods for intelligent identification of fall events based on medical data.

### BACKGROUND

Falls among elderly population are one leading cause of disability and mortality. Each year, more than $50 billion is spent on non-fatal fall injuries and $754 million is spent on fatal falls in the United States. On average, the hospitalization cost for a fall injury is $34,294, according to one example. As per the Centers for Disease Control and Prevention (CDC), falls are not a normal part of aging and may be prevented through pro-active interventions, for example. Falling once doubles the chances of falling again. Therefore, knowing the correct and complete fall history of the population may assist in identifying members at high risk of fall for proactive interventions. In medical claims, significant under coding of fall-related diagnosis codes has been observed.

The present disclosure is directed to overcoming one or more of the above-referenced challenges.

### SUMMARY OF THE DISCLOSURE

In some aspects, the techniques described herein relate to a method performed by one or more processors of a computing system, the method including: receiving at least one first data object for an entity; receiving at least one second data object for the entity, the at least one second data object being associated with the at least one first data object; determining if the at least one first data object includes at least one inclusionary code associated with a physical injury, the at least one inclusionary code being indicative of a fall-related injury; determining if the at least one first data object excludes at least one exclusionary code associated with a physical injury, the at least one exclusionary code being indicative of a non-fall-related injury; and when it is determined that the at least one first data object includes the at least one inclusionary code and excludes the at least one exclusionary code: marking the at least one first data object as being indicative of a possible fall event; determining if the at least one second data object includes at least one fall-related word; and when it is determined that the at least one second data object includes the at least one fall-related word, marking the at least one first data object as being indicative of an actual fall event.

In some aspects, the techniques described herein relate to a method, wherein the at least one inclusionary code and the at least one exclusionary code each include a diagnosis code.

In some aspects, the techniques described herein relate to a method, wherein the at least one inclusionary code includes a W-series ICD code or a S-series ICD code.

In some aspects, the techniques described herein relate to a method, wherein the at least one inclusionary code includes a R29.6 ICD-10 code.

In some aspects, the techniques described herein relate to a method, wherein the at least one exclusionary code includes a V-series ICD code, a W-series ICD code, an X-series ICD code, or a Y-series ICD code.

In some aspects, the techniques described herein relate to a method, wherein determining if the at least one second data object includes the at least one fall-related word includes performing a lexical search for the at least one fall-related word in the at least one second data object and satisfying a contextual match.

In some aspects, the techniques described herein relate to a method, wherein the at least one inclusionary code includes a diagnostic code associated with one or more diagnostic positions associated with the entity.

In some aspects, the techniques described herein relate to a method, wherein the at least one first data object includes historical medical claims data for the entity, the historical medical claims data spanning a duration data including: one or more historical fall events associated with one or more historical physical injuries, the one or more historical fall events including the actual fall event, the one or more historical fall events being identified based on inclusion of the at least one inclusionary code, exclusion of the at least one exclusionary code, and inclusion of the at least one fall-related word in the at least one second data object.

In some aspects, the techniques described herein relate to a method, further including: receiving informational data associated with the entity, the informational data including at least one of demographics data, diagnosis data, or medication data, wherein the demographics data includes data related to age, gender, income, or region of residence associated with the entity, wherein the diagnosis data includes data related to current or previous diagnoses associated with the entity, and wherein the medication data includes data related to current or previous medications prescribed to the entity; training a machine-learning model based on the one or more historical fall events and the informational data, the machine-learning model being configured to determine a relationship between the one or more historical fall events and the informational data; and determining if a different entity is likely to have a future fall event based on the machine-learning model.

In some aspects, the techniques described herein relate to a method, wherein determining if the at least one first data object includes the at least one inclusionary code and excludes the at least one exclusionary code includes performing a search lines of the at least one first data object to detect a presence of the at least one inclusionary code and detect an absence of the at least one exclusionary code.

In some aspects, the techniques described herein relate to a method, wherein when it is determined that the at least one second data object includes the at least one fall-related word, notifying the entity of an available medical intervention via a first computing device associated with the entity.

In some aspects, the techniques described herein relate to a method, wherein when it is determined that the at least one second data object includes the at least one fall-related word, modifying a third data object stored in a data store to include that the entity is associated with the actual fall event.

In some aspects, the techniques described herein relate to a method, wherein when it is determined that the at least one second data object includes the at least one fall-related word, transmitting a notification to a computing device associated with a healthcare provider that the entity has likely experienced the actual fall event.

In some aspects, the techniques described herein relate to a method, wherein the at least one first data object includes medical claims data and the at least one second data object includes medical note data.

In some aspects, the techniques described herein relate to a system for fall event detection, the system including: at least one memory having processor-readable instructions stored therein; and at least one processor configured to access the at least one memory and execute the processor-readable instructions to perform operations, the operations including: receiving at least one first data object for an entity; receiving at least one second data object for the entity, the at least one second data object being associated with the at least one first data object; determining if the at least one first data object includes at least one inclusionary code associated with a physical injury, the at least one inclusionary code being indicative of a fall-related injury; determining if the at least one first data object excludes at least one exclusionary code associated with a physical injury, the at least one exclusionary code being indicative of a non-fall-related injury; and when it is determined that the at least one first data object includes the at least one inclusionary code and excludes the at least one exclusionary code: marking the at least one first data object as being indicative of a possible fall event; determining if the at least one second data object includes at least one fall-related word; and when it is determined that the at least one second data object includes the at least one fall-related word, marking the at least one first data object as being indicative of an actual fall event.

In some aspects, the techniques described herein relate to a system, wherein determining if the at least one second data object includes the at least one fall-related word includes performing a lexical search for the at least one fall-related word in the at least one second data object and satisfying a contextual match.

In some aspects, the techniques described herein relate to a system, wherein determining if the at least one first data object includes the at least one inclusionary code and excludes the at least one exclusionary code includes performing a search lines of the at least one first data object to detect a presence of the at least one inclusionary code and detect an absence of the at least one exclusionary code.

In some aspects, the techniques described herein relate to a system, wherein when it is determined that the at least one second data object includes the at least one fall-related word, transmitting a notification to a computing device associated with a healthcare provider that the entity has likely experienced the actual fall event.

In some aspects, the techniques described herein relate to a system, wherein when it is determined that the at least one second data object includes the at least one fall-related word, notifying the entity of an available medical intervention via a first computing device associated with the entity.

In some aspects, the techniques described herein relate to a non-transitory computer-readable medium storing a set of instructions that, when executed by at least one processor, cause the at least one processor to perform operations including: receiving at least one first data object for an entity; receiving at least one second data object for the entity, the at least one second data object being associated with the at least one first data object; determining if the at least one first data object includes at least one inclusionary code associated with a physical injury, the at least one inclusionary code being indicative of a fall-related injury; determining if the at least one first data object excludes at least one exclusionary code associated with a physical injury, the at least one exclusionary code being indicative of a non-fall-related injury; and when it is determined that the at least one first data object includes the at least one inclusionary code and excludes the at least one exclusionary code: marking the at least one first data object as being indicative of a possible fall event; determining if the at least one second data object includes at least one fall-related word; and when it is determined that the at least one second data object includes the at least one fall-related word, marking the at least one first data object as being indicative of an actual fall event.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary system infrastructure for identifying fall events, according to one or more embodiments.

FIG. 2 depicts an exemplary flowchart of a method for identifying fall events and predicting fall events, according to one or more embodiments.

FIG. 3 depicts an exemplary flowchart of a method for identifying fall events, according to one or more embodiments.

FIG. 4 depicts an exemplary flowchart for identifying fall events according to one embodiment and another exemplary flowchart of a method for identifying fall events according another embodiment.

FIG. 5 depicts an exemplary machine-learning training flow chart, according to one or more embodiments.

FIG. 6 depicts an exemplary implementation of a computer system that executes techniques presented herein, according to one or more embodiments.

### DETAILED DESCRIPTION OF EMBODIMENTS

Various embodiments of the present disclosure relate generally to systems and methods for intelligent identification of fall events, and more particularly, to systems and methods for identifying a possible fall event by determining if at least one first data object includes at least one inclusionary code associated with a physical injury and excludes at least one exclusionary code associated with a physical injury and, when it is determined that the at least one first data object includes a possible fall event, determining if at least one second data object includes a fall-related word.

As previously discussed, falls among the elderly population (e.g., aged 65 years and above) are a leading cause of disability and mortality. Each year more than $50 billion is spent on non-fatal fall injuries and approximately $754 million is spent on fatal fall injuries in the United States. On average, hospitalization cost for a fall injury is approximately $34,294. As per the Centers for Disease Control and Prevention (CDC), falls are not a normal part of aging and may be prevented through pro-active interventions. Falling once may double chances of falling again. Knowing the correct fall history of the population may be helpful to identify members at high risk of fall for proactive interventions.

In some cases, diagnosis information found in medical claims may be an easily available source for a population's fall history generation. In some medical claims, however, under coding of fall-related diagnosis codes are generally observed. For example, under coding may mean that fall-related ICD 10 diagnosis codes (e.g., a subset of W00.xxx to W19.xxx codes) are not present for each fall-related encounter. This may be because of the conventional coding practices or because these codes are not used for reimbursement purposes. In fall-related medical claims, it has been observed that injury-related diagnosis codes (e.g., ICD 10 codes) were coded well in some instances, but fall-related ICD 10 diagnosis codes were missing in many cases. Implications of under coding for fall-related events may result in incomplete population fall history generation using medical claims data.

In various cases, under coding of fall-related diagnosis codes may occur for various medical claims when patients encounter a fall, go to see a physician, and the physician enters a S-series ICD 10 code (injuries to the knee and lower leg). In this scenario, the event likely should have been tagged as a W-series ICD 10 code to be properly identified as a fall event. In some cases, up to approximately two-thirds of fall events may not be coded properly in some medical claims data. In some cases, using a conventional approach of looking for fall-related diagnosis codes (a subset of W codes-W00.xxx to W19.xxx5 series and R29.6) can result in identification of only one third of all fall events. Some possible reasons for under coding include that prevailing coding practices or a majority of applicable codes are not used for reimbursement purposes.

However, in a large amount of cases, fall-related indicators and signals are found to be present in some other forms and manifestations (e.g., injury codes, medical notes, etc.) that may be used to accurately identify fall events.

As per ICD 10 code definition, fall events are conventionally defined using evidence of the subset of codes from W00.xxx to W19.xxx code series or R29.6 code at any diagnostic positions. However, as discussed previously, under coding of fall diagnosis codes occurs in the healthcare industry. Therefore, there may be other claims or encounters related to a fall which do not have the required W series ICD 10 or R29.6 diagnostic codes, but have injury-related ICD 10 codes.

To address the issues outlined above, one or more embodiments of the present disclosure include detecting non-obvious but clinically-related signals of falls in medical claims to identify fall events. One or more embodiments discussed herein identify possible and actual fall events based on inclusion and exclusion of certain inclusionary and exclusionary codes associated with injury diagnostic codes. One or more embodiments detect the clinically-related signals of falls by identifying evidence of falls using one or more inclusionary and exclusionary criteria (e.g., detecting inclusionary and exclusionary diagnostic codes in medical claims data) built around available diagnosis codes related to injuries, accidents (e.g., motor vehicle accidents), and other external causes of morbidity.

One or more embodiments include, when it is determined that medical claims data for an entity (e.g., healthcare patient) includes at least one inclusionary code and excludes at least one exclusionary code, contextually matching medical notes for the entity for fall-related words. Contextually matching includes using lexical searches for the medical notes to look for fall indicators. Determining if the medical claims data includes at least one inclusionary code and excludes at least one exclusionary code can help narrow down the context so that the lexical searches performed on medical notes may be more accurate, and the amount of data objects to be analyzed may be reduced, thus saving computing resources and accelerating data processing.

The approach outlined above offers various advantages over conventional methods of identifying fall events. One or more embodiments are computationally efficient due to the scope for identifying fall-related signals being narrowed by the identification of the at least one inclusionary and at least one exclusionary codes, and performing a contextual match in response to the identification of the at least one inclusionary and exclusionary codes.

As one skilled in the art will appreciate in light of this disclosure, certain embodiments may be capable of achieving certain advantages, including some or all of the following: (1) reducing computer resource utilization (e.g., memory consumption, processor utilization, network transfer, etc.) by reducing the number of data objects that may need to be analyzed for identifying fall events and increasing accuracy of identification of data objects that include actual fall events, by first preliminarily identifying if at least one data object includes at least one inclusionary code and excludes at least one exclusionary code associated with a physical injury, and based on the result of the preliminary identification step, determining if at least one second data object associated with the at least one first data object includes at least one fall-related word to identify an actual fall event; and (2) improving the functioning and reliability of a computing system through use of machine-learning models to predict future fall events, where the machine-learning models may be trained with the data objects marked as including actual fall events and other informational data associated with the entity, and where use of the machine-learning models with the marked data objects may improve the sensitivity and precision of the computing system developed for fall prevention.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

Various non-limiting embodiments of the present disclosure will now be described to provide an overall understanding of the principles of the structure, function, and use of systems and methods disclosed herein for intelligently identifying actual fall events for an entity.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of +10% of a stated or understood value.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine-learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

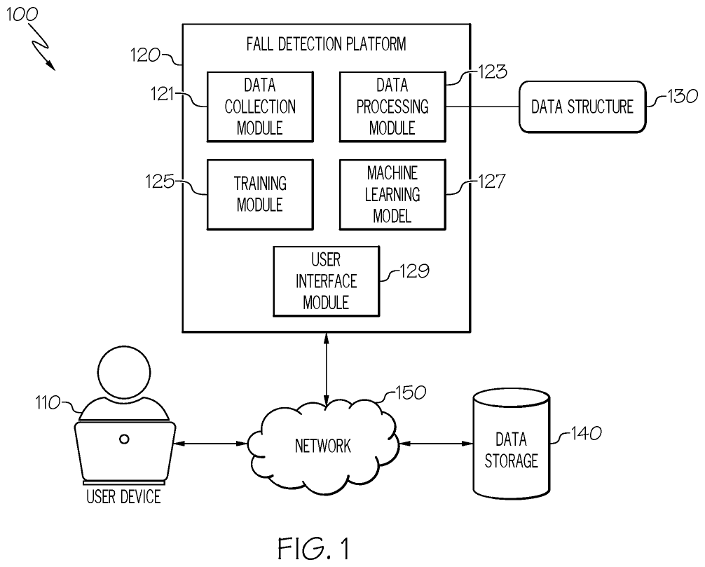

Referring now to the drawings, FIG. 1 depicts an example system 100 that is implemented with the techniques presented herein for identifying fall events, according to one or more embodiments. For example, the system 100 is used for fall event detection for various entities (e.g. healthcare patients, elderly healthcare patients, etc.), engaging in prevention interventions once actual fall events are detected, and predicting fall events by using machine-learning models. As depicted in FIG. 1, the system 100 includes a user device 110, a fall detection platform 120, a data store 140, and a network 150. The data store 140 may store various types of data related to patient information. For example, the data store 140 may store various data objects related to elderly and/or non-elderly healthcare patients including data objects such as medical claims data and medical notes data. The data store 140 may also store other data objects including informational data regarding patients such as demographics data, diagnosis data, and medication data. The demographics data, diagnosis data, and medication data may be included as part of the medical claims data, or may be stored as separate data objects in the data store 140 in some examples.

The medical claims data includes current and/or historical medical claims data associated with healthcare patients. Current medical claims data may include claims data that is recent (e.g., medical claims filed within past year, most recently filed medical claims, etc.). Historical medical claims data may include medical claims data spanning a patient's entire available medical history. In some cases, historical medical claims data may include claims data spanning a duration of three or more years for a patient.

The demographics data may include data related to age, gender, income, or region of residence associated with the patients. The diagnosis data may include data related to current or previous diagnoses associated with the patients. The medication data may include data related to current or previous medications prescribed to the patients.

In some examples, the data store 140 can include medical claims data, medical notes data, demographics data, diagnosis data, medication data, and other types of data for a given population (e.g., elderly patients over the age of 65). Medical claims data may be sourced from insurance providers and can include details regarding healthcare provider visits for a patient such as diagnosis (e.g., ICD-10 code, procedure (e.g., CPT codes), drug (e.g., medication), patient information (e.g., age, gender, location), payer (e.g., government/commercial insurance company responsible for payment), practitioner (e.g., HCPs responsible for rendering care), and facilities (e.g., location where services were provided), for example.

Medical notes data may also be sourced from insurance providers or HCPs, and may include notes written by a doctor or a nurse about a healthcare visit a patient has attended. For example, a specific medical note may be associated with a specific claim and may include further details about the nature of the injury not described in the claim (e.g., if a patient experienced a fall, the medical note may include descriptions about the injury and include a fall-related word somewhere in the note).

To identify possible fall events, including ones that may have been missed due to under coding or the like, the fall detection platform 120 is configured to identify a presence of certain inclusionary codes and an absence of certain exclusionary codes in the medical claims data. In this respect, the data store 140 is configured to store various inclusionary and exclusionary codes, as data objects, such as ICD 10 diagnosis codes, to be retrieved and used by the fall detection platform 120 to identify possible fall events. The exact nature of the inclusionary and exclusionary codes that are stored in the data store 140 to be used by the fall detection platform 120 may vary and be updated depending on coding practices in the healthcare industry and user preferences which possibly may change over time.

In some cases, the fall detection platform 120 is configured to perform searches for the inclusionary and exclusionary codes without having to retrieve the search terms from the data store 140. For example, the fall detection platform 120 may be configured to perform a contextual search or a search with predefined search queries for certain inclusionary and exclusionary codes based on user input.

System 100 is implemented on a network 150, allowing for the transmission or sharing of data between each user device 110, the fall detection platform 120, and the data store 140, in a networked environment. Some or all of the components illustrated in FIG. 1 may be implemented on a cloud platform.

The network 150 may include a wired and/or wireless network that may couple devices so that communications can be exchanged, such as between a server and a user device or other types of devices, including between wireless devices coupled via a wireless network, for example. A network can also include mass storage, such as network attached storage (NAS), a storage area network (SAN), or other forms of computer or machine-readable media, for example. A network can include the Internet, one or more local area networks (LANs), one or more wide area networks (WANs), wire-line type connections, wireless type connections, cellular or any combination thereof. Likewise, sub-networks, which can employ differing architectures or can be compliant or compatible with differing protocols, can interoperate within a larger network. Various types of devices can, for example, be made available to provide an interoperable capability for differing architectures or protocols. As one illustrative example, a router can provide a link between otherwise separate and independent LANs.

Furthermore, devices or user devices, such as computing devices or other related electronic devices can be remotely coupled to a network, such as via a wired or wireless line or link, for example.

In certain non-limiting embodiments, a “wireless network” should be understood to couple user devices with a network. A wireless network can include virtually any type of wireless communication mechanism by which signals can be communicated between devices, between or within a network, or the like. A wireless network can employ standalone ad-hoc networks, mesh networks, wireless land area network (WLAN), cellular networks, or the like. A wireless network may be configured to include a system of terminals, gateways, routers, or the like coupled by wireless radio links, or the like, which can move freely, randomly, or organize themselves arbitrarily, such that network topology can change, at times even rapidly.

A wireless network can further employ a plurality of network access technologies, including Wi-Fi, Long Term Evolution (LTE), WLAN, Wireless Router (WR) mesh, or 2nd, 3rd, 4th, 5th generation (2G, 3G, 4G, or 5G) cellular technology, or the like. Network access technologies can allow wide area coverage for devices, such as user devices with varying degrees of mobility, for example.

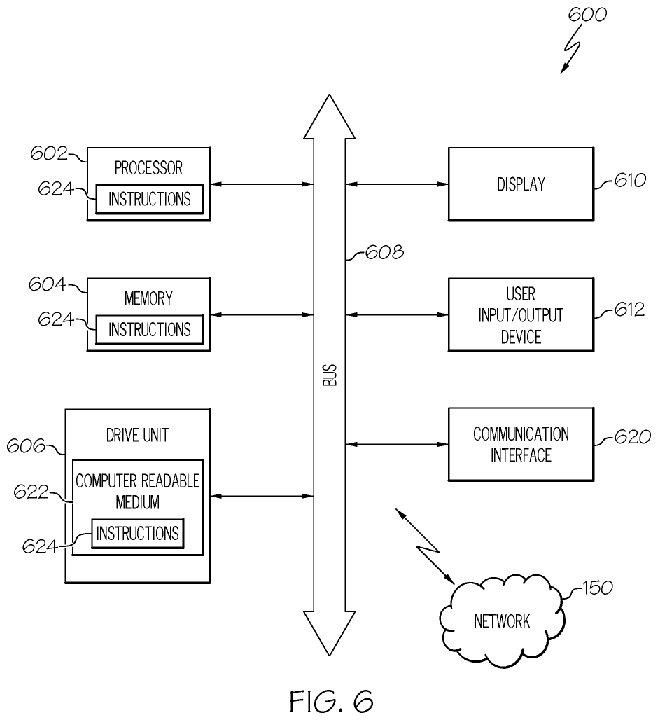

The user device 110 may include any electronic equipment, controlled by a processor (e.g., central processing unit (CPU)), for inputting information or data and displaying a user interface. A computing device or user device can send or receive signals, such as via a wired or wireless network, or can process or store signals, such as in memory as physical memory states. A user device may include, for example: a desktop computer; a mobile computer (e.g., a tablet computer, a laptop computer, or a notebook computer); a smartphone; a wearable computing device (e.g., smart watch); or the like, consistent with the computing devices shown in FIG. 6.

The user device 110 is used by a user (e.g., a patient, a healthcare provider (HCP) associated with the patient, etc.). When a fall event is identified by the fall detection platform 120, the fall detection platform 120 is configured to send a notification to the user device 110, alerting the user device 110 that a fall event was identified. In some cases, the fall detection platform 120 is configured to notify the user device 110 that medical interventions may be available to prevent further falls for the future. In some cases, the fall detection platform 120 may be configured to notify the user device 110 that a future fall event may be likely based on predictions generated by one or more machine-learning models.

The fall detection platform 120 provides certain modules such as a data collection module 121, a data processing module 123, a training module 125, a machine-learning model 127 and a user interface module 129, and/or the like for performing certain tasks, such as identifying possible and actual fall events, notifying the user device 110 of actual fall events that have been identified, and predicting likely fall events for one or more patients.

The data collection module 121 is configured to receive the data objects stored in the data store 140, such as medical claims data, medical notes data, demographics data, diagnosis data, and medication data for patients such as elderly patients, according to one example. In some cases, the data collection module 121 is configured to receive various inclusionary and exclusionary codes from the data store 140, so that the data processing module 123 may search the medical claims data to determine a presence of certain inclusionary codes and an absence of certain exclusionary codes.

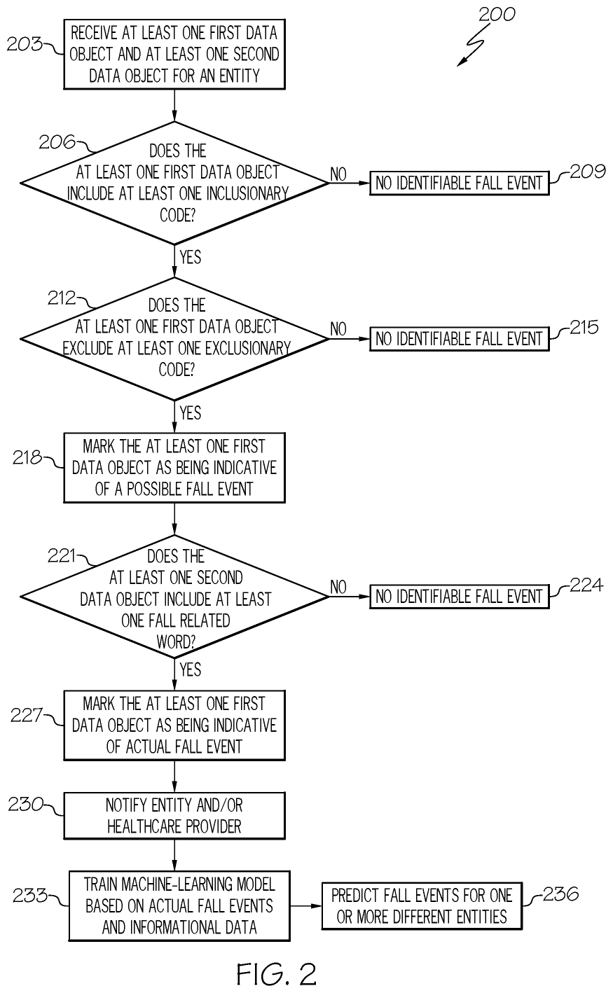

The data processing module 123 is configured to process the data collected by the data collection module 121 to identify possible fall events and actual fall events. Identifying or determining a possible fall event for a patient includes determining if medical claims data includes at least one inclusionary code associated with a physical injury for a patient and excludes at least one exclusionary code associated with a physical injury for a patient. For example, the data processing module 123 may perform a search of the medical claims data to identify individual claim lines for a presence and/or absence of certain inclusionary and exclusionary codes, respectively. Further description regarding specific inclusionary and exclusionary codes which may be used to determine a possible fall event is discussed in greater detail with respect to the flowchart in FIG. 2.

As discussed previously, the data store 140 is configured to store various inclusionary and exclusionary codes, as data objects, such as ICD 10 diagnosis codes, to be retrieved and used by the fall detection platform 120 to identify possible fall events. The exact nature of the inclusionary and exclusionary codes that are stored in the data store 140 to be used by the fall detection platform 120 may vary and be updated depending on coding practices in the healthcare industry and user preferences, which may change over time. In some cases, the fall detection platform 120 may perform searches for the inclusionary and exclusionary codes without having to retrieve the search terms from the data store 140. For example, the fall detection platform 120 may be programmed to perform a contextual search or a search with predefined search queries for certain inclusionary and exclusionary codes based on user input.

When it is determined that the medical claims data for a patient includes a possible fall event, the data processing module 123 determines if the medical claims data includes an actual fall event by performing a word search of the medical notes data to contextually search for a fall-related word. For example, a “fall”-related word can include the word “fall,” “fell,” and/or “fallen,” among others. The data processing module 123 may search a portion of the medical notes data that is associated with a specific claim line of the medical claims data to determine a contextual match of a fall-related word. A lexical search of the medical notes data for the “fall”-related word can produce accurate results, as the scope of the search is focused on portions of the medical notes data associated with the medical claims data that includes the certain inclusionary code(s) and excludes the certain exclusionary code(s).

In some embodiments, the data processing module 123 generates a data structure 130. The data structure 130 may include results of the possible fall event identification step and/or the results of the possible and actual fall identification steps. For example, if a possible fall event is identified, the medical claims data may be updated for a respective patient to reflect this outcome. If an actual fall event is identified, the medical claims data may also be updated for the respective patient to reflect this outcome. In at least one outcome, the data structure 130 may include a table. In some examples, the data structure 130 is stored in the data store 140.

The training module 125 may provide learning, or training to the machine-learning model 127 by providing training data, e.g., data from other modules that contains inputs (e.g., stage inputs) and known outcomes, to allow the machine-learning model 127 to learn over time. For example, the training module 125 may receive data from the data structure 130 generated by the data processing module 123 and data from the data store 140 and provide the received data to the machine-learning model 127. The training module 125 may conduct the training in any suitable manner, e.g., in batches, and may include any suitable training methodology. Training may be performed periodically, and/or continuously, e.g., in real-time or near real-time. Further details of training a machine-learning model are provided below.

Machine-learning model 127 receives the training data from the training module 125 to learn relationships between actual fall events that have been identified for a patient and the patient's informational data (e.g., diagnosis data, demographics data, medication data, etc.). For example, the machine-learning model 127 uses programmed algorithms for identifying the above-mentioned relationships. The machine-learning model 127 is configured to identify which patients are likely to experience a fall event in the future or experience a repeat fall event, among other possible outcomes based on the learned relationships. The ordering of the training data may be randomized during training. The machine-learning model 127 may visualize the training data to identify relevant relationships between different variables and identify any data imbalances. The training data may be split into two parts where one part is for training the model and the other part is for validating the trained model, de-duplicating, normalizing, correcting errors in the training data, and so on. In some examples, the machine-learning model 127 receives data directly from the data structure 130. The machine-learning model 127 may implement various machine-learning techniques (e.g., random forest, k-nearest neighbor, partial least squares regression, principal component regression, etc.) discussed in the present disclosure.

The user interface module 129 enables a presentation of a graphical user interface (GUI) in the user device 110. The user interface module 129 includes a variety of interfaces, for example, interfaces for data input and output devices, referred to as I/O devices, storage devices, and the like.

FIG. 2 depicts an exemplary method 200 for identifying fall events and predicting fall events, according to one or more embodiments. The method 200 is used for identifying possible and actual fall events for various entities (e.g., healthcare patients) and predicting fall events based on use of one or more machine-learning models. The fall detection platform 120 is configured to implement the method 200 according to various embodiments.

At step 203, the fall detection platform 120 receives at least one first data object and at least one second data object for an entity. The at least one first data object corresponds to medical claims data sourced from insurance providers, healthcare providers, etc. The at least one second data object corresponds to medical notes data that is associated with the medical claims data. The at least one first data object and the at least one second data object are stored in the data store 140 and are retrievable by the fall detection platform 120.

For example, the medical claims data stored in the data store 140 may include medical claims data for elderly population patients over the age of 65. The medical notes data includes notes written by a doctor or nurse associated with the medical claims data for which a patient has attended a healthcare visit. A specific medical note may be associated with a specific claim and may include further details about the nature of the injury not described in the claim (e.g., if a patient experienced a fall, the medical note may include descriptions about the injury and include a fall-related word in the note).

At step 206, the fall detection platform 120 is configured to identify possible fall events. Possible fall events are identified based on searches through medical claims data to determine if the medical claims data includes certain inclusionary codes and excludes certain exclusionary codes associated with a physical injury for a patient.

To identify possible fall events, including ones that may have been missed due to under coding or the like, the fall detection platform 120 is configured to identify a presence of certain inclusionary codes and identify an absence of certain exclusionary codes associated with a physical injury in the medical claims data. As per ICD 10 code definition, fall events are conventionally defined using evidence of the subset of codes from W00.xxx to W19.xxx code series or R29.6 code at any diagnostic positions. However, as discussed previously, under coding of fall diagnosis codes occurs in the healthcare industry. Therefore, there may be other claims or encounters related to a fall which do not have the required W series ICD 10 or R29.6 diagnostic codes, but have injury-related ICD 10 codes. Thus, the embodiments discussed herein identify possible and actual fall events based on inclusion and exclusion of certain inclusionary and exclusionary codes associated with injury diagnostic codes.

To identify possible fall events, at step 206, the fall detection platform 120 is configured to search for a presence of W series ICD 10 codes ranging from W00.xxx to W19.xxx, certain S series ICD 10 codes, or a R29.6 ICD 10 code, at any diagnostic positions in the medical claims data. Tables 1 and 2 describe exemplary W series ICD 10 codes, S series ICD 10 codes, or a R29.6 ICD 10 codes that the fall detection platform 120 can search for.

TABLE 1
W series ICD 10 codes and R29.6 ICD 10 code

| ICD 10 Code | |
|---|---|
| series | Description |
| W19.xxx | Unspecified fall |
| W18.xxx | Other slipping, tripping and stumbling and falls (W18) |
| W01.xxx | Fall on same level from slipping, tripping and stumbling |
| W10.xxx | Fall on and from stairs and steps |
| W06.xxx | Fall from bed |
| W07.xxx | Fall from chair |
| W11.xxx | Fall on and from ladder |
| W17.xxx | Other fall from one level to another |
| W05.xxx | Fall from non-moving wheelchair, non-motorized scooter |
| | and motorized mobility scooter |
| W08.xxx | Fall from other furniture |
| R29.6 | Repeated falls |

TABLE 2
S series ICD 10 codes

| ICD 10 Code | |
|---|---|
| series | Description |
| S00-S09 | Injuries to the head |
| S80-S89 | Injuries to the knee and lower leg |
| S30-S39 | Injuries to the abdomen, lower back, lumbar spine, pelvis |
| | and external genitals |
| S90-S99 | injuries to the ankle and foot |
| S40-S49 | Injuries to the shoulder and upper arm |
| S20-S29 | Injuries to the thorax |
| S60-S69 | Injuries to the wrist, hand and fingers |
| S70-S79 | Injuries to the hip and thigh |
| S10-S19 | injuries to the neck |
| S50-S59 | Injuries to the elbow and forearm |

The W series ICD 10 codes in Table 1 correspond to a first inclusionary criteria the fall detection platform 120 searches an inclusion for in the medical claims data. The S series ICD 10 codes in Table 2 correspond to a second inclusionary criteria the fall detection platform 120 searches an inclusion for in the medical claims data. If the fall detection platform 120 detects any of the codes described above in any claim line in the medical claims data, the method 200 moves to step 212 as preliminarily identifying a possible fall event. If the fall detection platform 120 does not detect any of the codes described above in any claim line in the medical claims data, then the method 200 moves to step 209. At step 209, no identifiable fall event is detected in the medical claims data, and the method 200 ends.

At step 212, the fall detection platform 120 is configured to search for an absence of one or more exclusionary codes in any claim lines in the medical claims data to identify a possible fall event. The one or more exclusionary codes include certain V series ICD 10 code, certain W series ICD 10 codes, certain X series ICD 10 codes, and certain Y series ICD 10 codes. The one or more exclusionary codes indicate a non-fall-related injury. The fall detection platform 120 searches for an absence of these codes to identify possible fall events. For example, if a claim line in the medical claims data 303 includes any of the inclusionary codes from Table 1 but not any exclusionary codes from Table 3 or Table 4, then that claim line is labeled as a fall evidence claim line. In another example, if any claim line has any of the ICD 10 codes from Table 2 but not any exclusionary codes from Table 3 or Table 4, then that claim line is labeled as a fall evidence claim line. In a further example, if any claim line includes diagnosis code “R29.6” but not any exclusionary codes from Table 3 or Table 4, then that claim line is also labeled as a fall evidence claim line. The fall detection platform 120 is configured to combine all claim lines labeled as fall evidence claim lines to generate a fall evidence claim line data set as part of the data structure 130.

The exclusionary codes discussed above are used to rule out detection of injuries like fractures or sprains that may be caused by motor vehicle collisions or other external causes of injuries/morbidities other than fall events. Tables 3 and 4 below describe the exemplary exclusionary codes that the fall detection platform 120 searches for in the medical claims data:

TABLE 3
V series ICD 10 codes

| ICD 10 Code | |
|---|---|
| series | Description |
| V00-V09 | Pedestrian injured in transport accident |
| V10-V19 | Pedal cycle rider injured in transport accident |
| V20-V29 | Motorcycle rider injured in transport accident |
| V30-V39 | Occupant of three-wheeled motor vehicle injured in |
| | transport accident |
| V40-V49 | Car occupant injured in transport accident |
| V50-V59 | Occupant of pick-up truck or van injured in transport |
| | accident |
| V60-V69 | Occupant of heavy transport vehicle injured in transport |
| | accident |
| V70-V79 | Bus occupant injured in transport accident |
| V80-V89 | Other land transport accidents |
| V90-V94 | Water transport accidents |
| V95-V97 | Air and space transport accidents |
| V98-V99 | Other and unspecified transport accidents |

TABLE 4
W series, X series, and Y series ICD 10 codes

| ICD 10 Code | |
|---|---|
| series | Description |
| W20-W49 | Exposure to inanimate mechanical forces |
| W50-W64 | Exposure to animate mechanical forces |
| W65-W74 | Accidental non-transport drowning and submersion |
| W85-W99 | Exposure to electric current, radiation and extreme ambient |
| | air temperature and pressure |
| X00-X08 | Exposure to smoke, fire and flames |
| X10-X19 | Contact with heat and hot substances |
| X30-X39 | Exposure to forces of nature |
| X50-X50 | Overexertion and strenuous or repetitive movements |
| X52-X58 | Accidental exposure to other specified factors |
| X71-X83 | Intentional self-harm |
| X92-Y09 | Assault |
| Y21-Y33 | Event of undetermined intent |
| Y35-Y38 | Legal intervention, operations of war, military operations, |
| | and terrorism |
| Y62-Y69 | Misadventures to patients during surgical and medical care |
| Y70-Y82 | Medical devices associated with adverse incidents in |
| | diagnostic and therapeutic use |
| Y83-Y84 | Surgical and other medical procedures as the cause of |
| | abnormal reaction of the patient, or of later complication, |
| | without mention of |
| Y90-Y99 | Supplementary factors related to causes of morbidity |
| | classified elsewhere |

The V series ICD 10 codes in Table 3 correspond to a first exclusionary criteria the fall detection platform 120 searches an absence for in the medical claims data. The W series, X series, and Y series ICD 10 codes in Table 4 correspond to a second exclusionary criteria the fall detection platform 120 searches an absence for in the medical claims data. If the medical claims data includes any one of the exclusionary codes described above, the method 200 moves to step 215. At step 215, no identifiable fall event is detected in the medical claims data, and the method 200 ends. If the fall detection platform 120 does not detect any of the exclusionary codes described above in the medical claims data, then the fall detection platform 120 identifies that possible fall events exist in the medical claims data, and the method 200 moves to step 218.

At step 218, the fall detection platform 120 marks or updates the medical claims data as being indicative of containing possible fall events. For example, the fall detection platform 120 is configured to update the medical claims data in the data store 140 to indicate that a specific patient has experienced a possible fall event that is associated with a medical claim. In another example, the fall detection platform 120 generates a fall evidence claim line data set that combines the fall evidence claim lines identified in step 212, to identify patients who have experienced possible fall events. In some cases, the fall detection platform 120 is configured to generate a data structure 130 based on the updated medical claims data and/or the fall event data set, which may be used by the training module 125 to train the machine-learning model 127. Thereafter, the method 200 moves to step 221.

At step 221, the fall detection platform 120 is configured to retrieve at least one second data object (e.g., medical notes data) for the patients identified in the fall evidence claim line dataset generated in step 218. Contextual matching is performed on the medical notes data 327 for any fall-related words (e.g., “fall,” “fell,” and/or “fallen,” etc.). If a positive match is identified, the method 200 moves to step 227. If the fall detection platform 120 does not identify a fall-related word in the medical notes data, the method moves to step 224. At step 224, no identifiable fall event is detected based on the medical claims data and the medical notes data, and the method 200 ends.

At step 227, the fall detection platform 120 marks or updates the medical claims data as being indicative of containing actual fall events. For example, the fall detection platform 120 is configured to update the medical claims data in the data store 140 to indicate that a specific patient has experienced an actual fall event that is associated with a medical claim. In some cases, the fall detection platform 120 updates the fall evidence claim line data set created in step 218 to flag patients who have been identified as having actual fall events. In some cases, the fall detection platform 120 is configured to generate a data structure 130 based on the updated medical claims data and/or the fall evidence claim line data set, which may be used by the training module 125 to train the machine-learning model 127. Thereafter, the method 200 moves to step 230.

At step 230, the fall detection platform 120 notifies the user device 110 (e.g., healthcare provider, insurance provider, patient, etc.) that actual fall events have been identified. For example, for a patient flagged as having history of an actual fall event, the fall detection platform 120 is configured to generate actual fall event identification results on a display of the user device 110 (e.g., user device for healthcare provider associated with the patient for which actual fall event was identified, user device for insurance provider associated with the claim in which actual fall event was identified, and/or user device for the patient). In some cases, the user device 110 is configured to access the results through a web-based portal or through a healthcare application installed on the user device 110. Once a patient is identified and/or flagged as experiencing an actual fall event, the fall detection platform 120 may perform further assessment of the fall risk of the patient to identify potential root causes that led to the actual fall event. Based on the results received by the user device 110, the healthcare provider and/or the insurance provider associated with the patient may engage in fall prevention interventions with the patient to prevent future fall events. Examples of proactive health interventions that may be implemented for the patient include physical therapy for muscle weakness, medication review where polypharmacy may be the root cause, classroom teaching sessions to educate patients to be aware of fall risk reducing proactive measures, and home hazard assessments affecting fall risk, among other proactive health interventions. Thereafter, the method 200 moves to step 233.

At step 233, the fall detection platform 120 is configured to train the machine-learning model 127 with the data objects that have been marked as being indicative of actual fall events in step 227 along with informational data for the identified patients (e.g., demographics data, medication data, diagnosis data, etc.). The machine-learning model 127 receives the training data from the training module 125 to learn relationships between actual fall events that have been identified for a patient and the patient's informational data. The ordering of the training data may be randomized during training. The machine-learning model 127 may visualize the training data to identify relevant relationships between different variables and identify any data imbalances. The training data may be split into two parts where one part is for training the model and the other part is for validating the trained model, de-duplicating, normalizing, correcting errors in the training data, and so on. In some examples, the machine-learning model 127 receives data directly from the data structure 130. Thereafter, the method 200 moves to step 236.

At step 236, the fall detection platform 120 predicts likely fall events for various patients based on the learned relationships. For example, the machine-learning model 127 is configured to identify, in response to receiving informational data associated with patients, which patients are likely to experience a fall event in the future or experience a repeat fall event, among other possible outcomes based on the learned relationships. Similarly with step 230, once a patient is identified and/or flagged as likely to experience a fall event, the fall detection platform 120 may perform further assessment of the fall risk of the patient to identify potential root causes that led to the determination of being likely to experience a fall event. The healthcare provider and/or the insurance provider associated with the identified patient may engage in fall prevention interventions with the patient to prevent future fall events. Examples of proactive health interventions that may be implemented for the patient include physical therapy for muscle weakness, medication review where polypharmacy may be the root cause, classroom teaching sessions to educate patients to be aware of fall risk reducing proactive measures, and home hazard assessments which may affect fall risk, among other proactive health interventions. Thereafter, the method 200 ends.

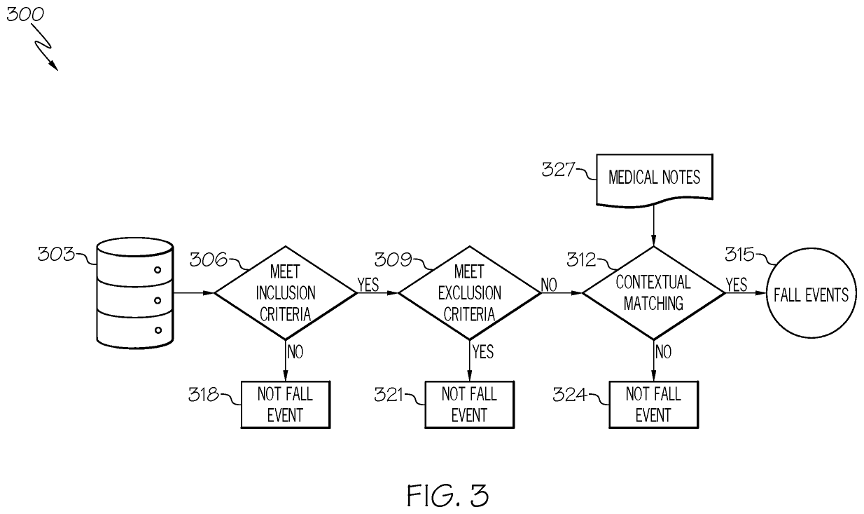

FIG. 3 depicts an exemplary method 300 for identifying fall events, according to various embodiments. The method 300 is used for identifying possible and actual fall events for various entities (e.g., healthcare patients). The fall detection platform 120 is configured to implement the method 300 according to various embodiments. The method 300 includes many of the same components and steps as the steps 203, 206, 209, 212, 215, 218, 221, 224, and 227 from method 200.

Starting with step 306, the fall detection platform 120 is configured to identify possible fall events based on medical claims data 303. Possible fall events are identified based on searches through the medical claims data 303 to determine if the medical claims data 303 includes certain inclusionary codes and excludes certain exclusionary codes associated with a physical injury for a patient.

To identify possible fall events, including ones that may have been missed due to under coding or the like, the fall detection platform 120 is configured to identify a presence of certain inclusionary codes and identify an absence of certain exclusionary codes associated with a physical injury in the medical claims data 303. As per ICD 10 code definition, fall events are defined using evidence of the subset of codes from W00.xxx to W19.xxx code series or R29.6 code at any diagnostic positions. However, as discussed previously, under coding of fall diagnosis codes occurs in the healthcare industry. Therefore, there may be other claims or encounters related to a fall which do not have the required W series ICD 10 or R29.6 diagnostic codes, but have injury-related ICD 10 codes.

To identify possible fall events, the fall detection platform 120 is configured to search for a presence of W series ICD 10 codes ranging from W00.xxx to W19.xxx, certain S series ICD 10 codes, or a R29.6 ICD 10 code, at any diagnostic positions in the medical claims data 303. Tables 1 and 2 listed above with respect to FIG. 2, describe exemplary W series ICD 10 codes, S series ICD 10 codes, or a R29.6 ICD 10 codes that the fall detection platform 120 can search for.

The W series ICD 10 codes in Table 1 correspond to a first inclusionary criteria the fall detection platform 120 searches an inclusion for in the medical claims data 306. The S series ICD 10 codes in Table 2 correspond to a second inclusionary criteria the fall detection platform 120 searches an inclusion for in the medical claims data 306. If the fall detection platform 120 detects any of the codes described above in any claim line in the medical claims data 306, the method 300 moves to step 309 as preliminarily identifying a possible fall event. If the fall detection platform 120 does not detect any of the codes described above in any claim line in the medical claims data 30, then the method 300 moves to step 318. At step 318, no identifiable fall event is detected in the medical claims data 303, and the method 300 ends.

At step 309, the fall detection platform 120 is configured to search for an absence of one or more exclusionary codes in any claim lines in the medical claims data 303 to identify a possible fall event. The one or more exclusionary codes include certain V series ICD 10 code, certain W series ICD 10 codes, certain X series ICD 10 codes, and certain Y series ICD 10 codes. The fall detection platform 120 searches for an absence of these codes to identify possible fall events. For example, if a claim line in the medical claims data 303 includes any of the inclusionary codes from Table 1 but not any exclusionary codes from Table 3 or Table 4, then that claim line is labeled as a fall evidence claim line. In another example, if any claim line has any of the ICD 10 codes from Table 2 but not any exclusionary codes from Table 3 or Table 4, then that claim line is labeled as a fall evidence claim line. In a further example, if any claim line includes diagnosis code “R29.6” but not any exclusionary codes from Table 3 or Table 4, then that claim line is also labeled as a fall evidence claim line. The fall detection platform 120 is configured to combine all claim lines labeled as fall evidence claim lines to generate a fall evidence claim line data set as part of the data structure 130.

The exclusionary codes discussed above are used to rule out detection of injuries like fractures or sprains that may be caused by motor vehicle collisions or other external causes of injuries/morbidities other than fall events. Tables 3 and 4 listed above with respect to FIG. 2 describe the exemplary exclusionary codes that the fall detection platform 120 searches for in the medical claims data 303.

The V series ICD 10 codes in Table 3 correspond to a first exclusionary criteria the fall detection platform 120 searches an absence for in the medical claims data 303. The W series, X series, and Y series ICD 10 codes in Table 4 correspond to a second exclusionary criteria the fall detection platform 120 searches an absence for in the medical claims data 303. If the medical claims data 303 includes any one of the exclusionary codes described above, the method 200 moves to step 321. At step 321, no identifiable fall event is detected in the medical claims data 303, and the method 200 ends. If the fall detection platform 120 does not detect any of the exclusionary codes described above in the medical claims data 303, then the fall detection platform 120 identifies that possible fall events exist in the medical claims data 303, and the method 300 moves to step 312.

At step 312, the fall detection platform 120 is configured to retrieve medical notes data 327 for the patients identified in the fall evidence claim line dataset generated in step 309. Contextual matching is performed on the medical notes data 327 for any fall-related words (e.g., “fall,” “fell,” and/or “fallen,” etc.). If a positive match is identified, the method 300 moves to step 315. If the fall detection platform 120 does not identify a fall-related word in the medical notes data 327, the method 300 moves to step 324. At step 324, no identifiable fall event is detected based on searches of the medical claims data 303 and the medical notes data 327, and the method 300 ends.

At step 315, the fall detection platform 120 marks or updates the medical claims data as being indicative of containing actual fall events. For example, the fall detection platform 120 is configured to update the medical claims data in the data store 140 to indicate that a specific patient has experienced an actual fall event that is associated with a medical claim. In some cases, the fall detection platform 120 updates the fall evidence claim line data set created in step 309 to flag patients who have been identified as having actual fall events. In some cases, the fall detection platform 120 is configured to generate a data structure 130 based on the updated medical claims data and/or the fall evidence claim line data set, which may be used by the training module 125 to train the machine-learning model 127. Thereafter, the method 300 ends.

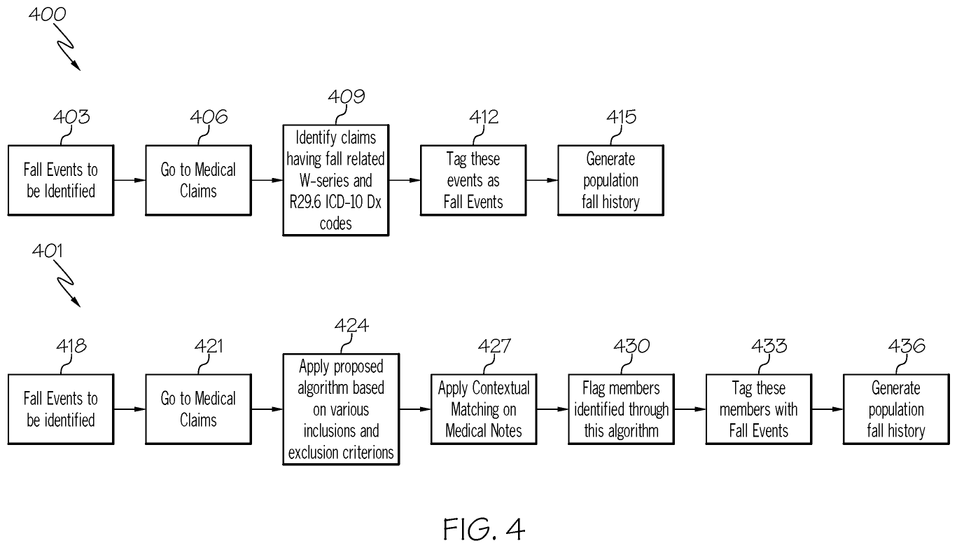

FIG. 4 depicts an exemplary flowchart for identifying fall events according to one embodiment and another exemplary flowchart of a method for identifying fall events according to another embodiment. Exemplary method 400 corresponds to a more simplified method of identifying fall events, and may be performed by the fall detection platform 120. In reference to step 409, fall events 403 for a population may generally be identified based on a search for inclusion of specified W series ICD 10 codes and R29.6 ICD 10 code in medical claims data 406. Claim lines in the medical claims data 406 that have been identified as including fall events based on inclusion of these codes may be tagged as including fall events at step 412. At step 415, a population fall history may be generated based on the tagged fall events identified in step 412. The population fall history generated at step 415 may include data objects containing a list of patients with identified (tagged and/or flagged) fall events and/or data objects containing portions of the medical claims data 406 associated with identified fall events. The portions of the medical claims data 406 included in the population fall history may include diagnosis (e.g., ICD-10 code, procedure (e.g., CPT codes), drug (e.g., medication), patient information (e.g., age, gender, location), payer (e.g., government/commercial insurance company responsible for payment), practitioner (e.g., HCPs responsible for rendering care), and facilities (e.g., location where services were provided) information for each claim line identified to be including a fall event. However, a disadvantage with the method 400 may be that only about one third (⅓^{rd}) of all fall events may be identified due to reasons previously discussed at paragraph 17, for example.

The method 401 is used for identifying possible and actual fall events for various entities (e.g., healthcare patients). The fall detection platform 120 is configured to implement the method 401 according to various embodiments. The method 401 includes many of the same components and steps as the steps 203, 206, 209, 212, 215, 218, 221, 224, and 227 from the method 200 and the steps 306, 309, 312, and 315 from the method 300. Steps 418, 421, 430, 433, and 436 are similar to the steps 403, 406, 409, 412, and 415 from method 400.

Starting with step 418, the fall detection platform 120 is configured to identify possible fall events 418 based on medical claims data 421. Possible fall events are identified based on searches through the medical claims data 421 to determine if the medical claims data 421 includes certain inclusionary codes and excludes certain exclusionary codes associated with a physical injury for a patient.

To identify possible fall events, as in step 424, including ones that may have been missed due to under coding or the like, the fall detection platform 120 is configured to identify a presence of certain inclusionary codes and identify an absence of certain exclusionary codes associated with a physical injury in the medical claims data 306. As per ICD 10 code definition, fall events are defined using evidence of the subset of codes from W00.xxx to W19.xxx code series or R29.6 code at any diagnostic positions. However, as discussed previously, under coding of fall diagnosis codes occurs in the healthcare industry. Therefore, there may be other claims or encounters related to a fall which do not have the required W series ICD 10 or R29.6 diagnostic codes, but have injury-related ICD 10 codes.

To identify possible fall events, the fall detection platform 120 is configured to search for a presence of W series ICD 10 codes ranging from W00.xxx to W19.xxx, certain S series ICD 10 codes, or a R29.6 ICD 10 code, at any diagnostic positions in the medical claims data 421. Tables 1 and 2 listed above with respect to FIG. 2, describe exemplary W series ICD 10 codes, S series ICD 10 codes, or a R29.6 ICD 10 codes that the fall detection platform 120 can search for.

The W series ICD 10 codes in Table 1 correspond to a first inclusionary criteria the fall detection platform 120 searches an inclusion for in the medical claims data 421. The S series ICD 10 codes in Table 2 correspond to a second inclusionary criteria the fall detection platform 120 searches an inclusion for in the medical claims data 421. If the fall detection platform 120 detects any of the codes described above in any claim line in the medical claims data 421, the fall detection platform 120 is configured to further search for an absence of one or more exclusionary codes in any claim lines in the medical claims data 421. The one or more exclusionary codes include certain V series ICD 10 code, certain W series ICD 10 codes, certain X series ICD 10 codes, and certain Y series ICD 10 codes. The fall detection platform 120 searches for an absence of these codes to identify possible fall events.

For example, if a claim line in the medical claims data 303 includes any of the inclusionary codes from Table 1 but not any exclusionary codes from Table 3 or Table 4, then that claim line is labeled as a fall evidence claim line. In another example, if any claim line has any of the ICD 10 codes from Table 2 but not any exclusionary codes from Table 3 or Table 4, then that claim line is labeled as a fall evidence claim line. In a further example, if any claim line includes diagnosis code “R29.6” but not any exclusionary codes from Table 3 or Table 4, then that claim line is also labeled as a fall evidence claim line. The fall detection platform 120 is configured to combine all claim lines labeled as fall evidence claim lines to generate a fall evidence claim line data set as part of the data structure 130.

The exclusionary codes discussed above are used to rule out detection of injuries like fractures or sprains that may be caused by motor vehicle collisions or other external causes of injuries/morbidities other than fall events. Tables 3 and 4 listed above with respect to FIG. 2 describe the exemplary exclusionary codes that the fall detection platform 120 searches for in the medical claims data 421.

The V series ICD 10 codes in Table 3 correspond to a first exclusionary criteria the fall detection platform 120 searches an absence for in the medical claims data 303. The W series, X series, and Y series ICD 10 codes in Table 4 correspond to a second exclusionary criteria the fall detection platform 120 searches an absence for in the medical claims data 303. If the medical claims data 303 includes any of the inclusionary codes and excludes all of the exclusionary codes described above, the method 401 moves to step 427.

At step 427, the fall detection platform 120 is configured to retrieve medical notes data for the patients identified in the fall evidence claim line dataset generated in step 424. Contextual matching is performed on the medical notes data for any fall-related words (e.g., “fall,” “fell,” and/or “fallen,” etc.). If a positive match is identified, the method 401 moves to step 430.

At steps 430 and 433, the fall detection platform 120 marks or updates the medical claims data 421 as being indicative of containing actual fall events. For example, the fall detection platform 120 is configured to update the medical claims data 421 in the data store 140 to indicate that a specific patient has experienced an actual fall event that is associated with a medical claim. In some cases, the fall detection platform 120 updates the fall evidence claim line data set created in step 424 to flag and/or tag patients who have been identified as having actual fall events. In some cases, the fall detection platform 120 is configured to generate a data structure 130 based on the updated medical claims data 421 and/or the fall evidence claim line data set, which may be used by the training module 125 to train the machine-learning model 127.

At step 436, the fall detection platform 120 may generate a population fall history based on the flagged and/or tagged patients who have been identified as having actual fall events. The population fall history generated at step 436 may include data objects containing a list of flagged and/or tagged patients with identified fall events and/or data objects containing portions of the medical claims data 421 associated with identified fall events. The portions of the medical claims data 421 included in the population fall history may include diagnosis (e.g., ICD-10 code, procedure (e.g., CPT codes), drug (e.g., medication), patient information (e.g., age, gender, location), payer (e.g., government/commercial insurance company responsible for payment), practitioner (e.g., HCPs responsible for rendering care), and facilities (e.g., location where services were provided) information for each claim line identified to be including a fall event, among others. The fall detection platform 120 may use the generated population fall history to train the machine-learning model 127. The machine-learning model 127 may use the generated population fall history along with the data contained in the data structure 130 and/or other data in the data store 140 to predict future fall events for various patients. Thereafter, the method 401 ends.

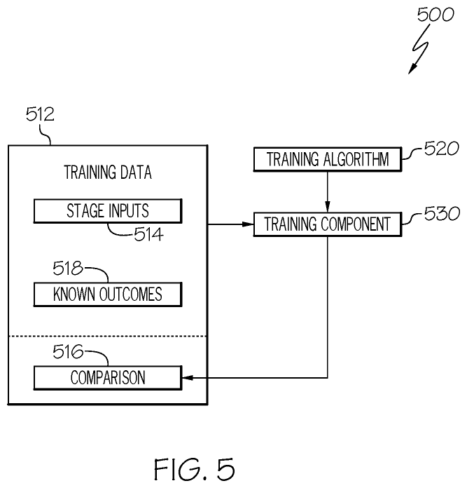

FIG. 5 depicts an exemplary machine-learning training flow chart, according to one or more embodiments. Referring to FIG. 5, a given machine-learning model (e.g., the machine-learning model 127) is trained using the training flow chart 500. The training data 512 includes one or more of stage inputs 514 and the known outcomes 518 related to the machine-learning model to be trained. The stage inputs 514 are from any applicable source including text, visual representations, data, values, comparisons, and stage outputs, e.g., one or more outputs from one or more steps from FIGS. 2-4. The known outcomes 518 are included for the machine-learning models generated based on supervised or semi-supervised training, or can based on known labels, such as topic labels. An unsupervised machine-learning model is not trained using the known outcomes 518. The known outcomes 518 includes known or desired outputs for future inputs similar to or in the same category as the stage inputs 514 that do not have corresponding known outputs.

The training data 512 and a training algorithm 520, e.g., one or more of the modules implemented using the machine-learning model and/or are used to train the machine-learning model, is provided to a training component 530 that applies the training data 512 to the training algorithm 520 to generate the machine-learning model. According to an implementation, the training component 530 is provided comparison results 516 that compare a previous output of the corresponding machine-learning model to apply the previous result to re-train the machine-learning model. The comparison results 516 are used by the training component 530 to update the corresponding machine-learning model. The training algorithm 520 utilizes machine-learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, the model specifically discussed herein, or the like.

The machine-learning model used herein is trained and/or used by adjusting one or more weights and/or one or more layers of the machine-learning model. For example, during training, a given weight is adjusted (e.g., increased, decreased, removed) based on training data or input data. Similarly, a layer is updated, added, or removed based on training data/and or input data. The resulting outputs are adjusted based on the adjusted weights and/or layers.

In general, any process or operation discussed in this disclosure is understood to be computer-implementable, such as the process illustrated in FIGS. 2-4, as being performed by one or more processors of a computer system as described herein. A process or process step performed by one or more processors is also referred to as an operation. The one or more processors are configured to perform such processes by having access to instructions (e.g., software or computer-readable code) that, when executed by one or more processors, cause one or more processors to perform the processes. The instructions are stored in a memory of the computer system. A processor is a central processing unit (CPU), a graphics processing unit (GPU), or any suitable type of processing unit.

A computer system, such as a system or device implementing a process or operation in the examples above, includes one or more computing devices. One or more processors of a computer system are included in a single computing device or distributed among a plurality of computing devices. One or more processors of a computer system are connected to a data storage device. A memory of the computer system includes the respective memory of each computing device of the plurality of computing devices.

FIG. 6 depicts an exemplary implementation of a computer system that executes techniques presented herein, according to one or more embodiments. The computer system 600 includes a set of instructions that are executed to cause the computer system 600 to perform any one or more of the methods or computer based functions disclosed herein. The computer system 600 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 600 operates in the capacity of a server or as a client user computer in a server-client user network environment, or as a peer computer system in a peer-to-peer (or distributed) network environment. The computer system 600 is also implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 600 is implemented using electronic devices that provide voice, video, or data communication. Further, while the computer system 600 is illustrated as a single system, the term “system” shall also be taken to include any collection of systems or sub-systems that individually or jointly execute a set, or multiple sets, of instructions to perform one or more computer functions.

As illustrated in FIG. 6, the computer system 600 includes a processor 602, e.g., a central processing unit (CPU), a graphics processing unit (GPU), or both. The processor 602 is a component in a variety of systems. For example, the processor 602 is part of a standard personal computer or a workstation. The processor 602 is one or more processors, digital signal processors, application specific integrated circuits, field programmable gate arrays, servers, networks, digital circuits, analog circuits, combinations thereof, or other now known or later developed devices for analyzing and processing data. The processor 602 implements a software program, such as code generated manually (i.e., programmed).

The computer system 600 includes a memory 604 that communicates via bus 608. The memory 604 is a main memory, a static memory, or a dynamic memory. The memory 604 includes, but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 604 includes a cache or random-access memory for the processor 602. In alternative implementations, the memory 604 is separate from the processor 602, such as a cache memory of a processor, the system memory, or other memory. The memory 604 is an external storage device or database for storing data. Examples include a hard drive, compact disc (“CD”), digital video disc (“DVD”), memory card, memory stick, floppy disc, universal serial bus (“USB”) memory device, or any other device operative to store data. The memory 604 is operable to store instructions executable by the processor 602. The functions, acts, or tasks illustrated in the figures or described herein are performed by the processor 602 executing the instructions stored in the memory 604. The functions, acts, or tasks are independent of the particular type of instruction set, storage media, processor, or processing strategy and are performed by software, hardware, integrated circuits, firmware, micro-code, and the like, operating alone or in combination. Likewise, processing strategies include multiprocessing, multitasking, parallel processing, and the like.

As shown, the computer system 600 further includes a display 610, such as a liquid crystal display (LCD), an organic light emitting diode (OLED), a flat panel display, a solid-state display, a cathode ray tube (CRT), a projector, a printer or other now known or later developed display device for outputting determined information. The display 610 acts as an interface for the user to see the functioning of the processor 602, or specifically as an interface with the software stored in the memory 604 or in the drive unit 606.

Additionally or alternatively, the computer system 600 includes an input/output device 612 configured to allow a user to interact with any of the components of the computer system 600. The input/output device 612 is a number pad, a keyboard, a cursor control device, such as a mouse, a joystick, touch screen display, remote control, or any other device operative to interact with the computer system 600.

The computer system 600 also includes the drive unit 606 implemented as a disk or optical drive. The drive unit 606 includes a computer-readable medium 622 in which one or more sets of instructions 624, e.g. software, is embedded. Further, the sets of instructions 624 embodies one or more of the methods or logic as described herein. The sets of instructions 624 resides completely or partially within the memory 604 and/or within the processor 602 during execution by the computer system 600. The memory 604 and the processor 602 also include computer-readable media as discussed above.

In some systems, computer-readable medium 622 includes the set of instructions 624 or receives and executes the set of instructions 624 responsive to a propagated signal so that a device connected to network 150 communicates voice, video, audio, images, or any other data over the network 150. Further, the sets of instructions 624 are transmitted or received over the network 150 via the communication port or interface 620, and/or using the bus 608. The communication port or interface 620 is a part of the processor 602 or is a separate component. The communication port or interface 620 is created in software or is a physical connection in hardware. The communication port or interface 620 is configured to connect with the network 150, external media, the display 610, or any other components in the computer system 600, or combinations thereof. The connection with the network 150 is a physical connection, such as a wired Ethernet connection, or is established wirelessly as discussed below. Likewise, the additional connections with other components of the computer system 600 are physical connections or are established wirelessly. The network 150 alternatively be directly connected to the bus 608.

While the computer-readable medium 622 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that causes a computer system to perform any one or more of the methods or operations disclosed herein. The computer-readable medium 622 is non-transitory, and may be tangible.

The computer-readable medium 622 includes a solid-state memory such as a memory card or other package that houses one or more non-volatile read-only memories. The computer-readable medium 622 is a random-access memory or other volatile re-writable memory. Additionally or alternatively, the computer-readable medium 622 includes a magneto-optical or optical medium, such as a disk or tapes or other storage device to capture carrier wave signals such as a signal communicated over a transmission medium. A digital file attachment to an e-mail or other self-contained information archive or set of archives is considered a distribution medium that is a tangible storage medium. Accordingly, the disclosure is considered to include any one or more of a computer-readable medium or a distribution medium and other equivalents and successor media, in which data or instructions are stored.

In an alternative implementation, dedicated hardware implementations, such as application specific integrated circuits, programmable logic arrays, and other hardware devices, is constructed to implement one or more of the methods described herein. Applications that include the apparatus and systems of various implementations broadly include a variety of electronic and computer systems. One or more implementations described herein implement functions using two or more specific interconnected hardware modules or devices with related control and data signals that are communicated between and through the modules, or as portions of an application-specific integrated circuit. Accordingly, the present system encompasses software, firmware, and hardware implementations.

Computer system 600 is connected to the network 150. The network 150 defines one or more networks including wired or wireless networks. The wireless network is a cellular telephone network, an 802.10, 802.16, 802.20, or WiMAX network. Further, such networks include a public network, such as the Internet, a private network, such as an intranet, or combinations thereof, and utilizes a variety of networking protocols now available or later developed including, but not limited to TCP/IP based networking protocols. The network 150 includes wide area networks (WAN), such as the Internet, local area networks (LAN), campus area networks, metropolitan area networks, a direct connection such as through a Universal Serial Bus (USB) port, or any other networks that allows for data communication. The network 150 is configured to couple one computing device to another computing device to enable communication of data between the devices. The network 150 is generally enabled to employ any form of machine-readable media for communicating information from one device to another. The network 150 includes communication methods by which information travels between computing devices. The network 150 is divided into sub-networks. The sub-networks allow access to all of the other components connected thereto or the sub-networks restrict access between the components. The network 150 is regarded as a public or private network connection and includes, for example, a virtual private network or an encryption or other security mechanism employed over the public Internet, or the like.

In accordance with various implementations of the present disclosure, the methods described herein are implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, and HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure is implemented using any appropriate techniques for implementing the functionality described herein. The disclosure is not limited to any particular programming language or operating system.

It should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention are practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications are made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims. The present disclosure furthermore relates to the following aspects.
Example 1. A method performed by one or more processors of a computing system, the method comprising: receiving at least one first data object for an entity; receiving at least one second data object for the entity, the at least one second data object being associated with the at least one first data object; determining if the at least one first data object includes at least one inclusionary code associated with a physical injury, the at least one inclusionary code being indicative of a fall-related injury; determining if the at least one first data object excludes at least one exclusionary code associated with a physical injury, the at least one exclusionary code being indicative of a non-fall-related injury; and when it is determined that the at least one first data object includes the at least one inclusionary code and excludes the at least one exclusionary code: marking the at least one first data object as being indicative of a possible fall event; determining if the at least one second data object includes at least one fall-related word; and when it is determined that the at least one second data object includes the at least one fall-related word, marking the at least one first data object as being indicative of an actual fall event.
Example 2. The method of Example 1, wherein the at least one inclusionary code and the at least one exclusionary code each include a diagnosis code.
Example 3. The method of any of the preceding examples, wherein the at least one inclusionary code includes a W-series ICD code or a S-series ICD code.
Example 4. The method of any of the preceding examples, wherein the at least one inclusionary code includes a R29.6 ICD-10 code.
Example 5. The method of any of the preceding examples, wherein the at least one exclusionary code includes a V-series ICD code, a W-series ICD code, an X-series ICD code, or a Y-series ICD code.
Example 6. The method of any of the preceding examples, wherein determining if the at least one second data object includes the at least one fall-related word includes performing a lexical search for the at least one fall-related word in the at least one second data object and satisfying a contextual match.
Example 7. The method of any of the preceding examples, wherein the at least one inclusionary code includes a diagnostic code associated with one or more diagnostic positions associated with the entity.
Example 8. The method of any of the preceding examples, wherein the at least one first data object includes historical medical claims data for the entity, the historical medical claims data spanning a duration of three or more years, the historical medical claims data including: one or more historical fall events associated with one or more historical physical injuries, the one or more historical fall events including the actual fall event, the one or more historical fall events being identified based on inclusion of the at least one inclusionary code, exclusion of the at least one exclusionary code, and inclusion of the at least one fall-related word in the at least one second data object.
Example 9. The method of Example 8, further comprising: receiving informational data associated with the entity, the informational data including at least one of demographics data, diagnosis data, or medication data, wherein the demographics data includes data related to age, gender, income, or region of residence associated with the entity, wherein the diagnosis data includes data related to current or previous diagnoses associated with the entity, and wherein the medication data includes data related to current or previous medications prescribed to the entity; training a machine-learning model based on the one or more historical fall events and the informational data, the machine-learning model being configured to determine a relationship between the one or more historical fall events and the informational data;
and determining if a different entity is likely to have a future fall event based on the machine-learning model.
Example 10. The method of any of Examples 1-7, wherein determining if the at least one first data object includes the at least one inclusionary code and excludes the at least one exclusionary code includes performing a search of one or more individual claim lines of the at least one first data object to detect a presence of the at least one inclusionary code and detect an absence of the at least one exclusionary code.
Example 11. The method of any of Examples 1-7 and 10, wherein when it is determined that the at least one second data object includes the at least one fall-related word, notifying the entity of an available medical intervention via a first computing device associated with the entity.
Example 12. The method of any of Examples 1-7, 10, and 11, wherein when it is determined that the at least one second data object includes the at least one fall-related word, modifying a third data object stored in a data store to include that the entity is associated with the actual fall event.
Example 13. The method of any of Examples 1-7 and 10-12, wherein when it is determined that the at least one second data object includes the at least one fall-related word, transmitting a notification to a computing device associated with a healthcare provider that the entity has likely experienced the actual fall event.
Example 14. The method of any of Examples 1-7 and 10-13, wherein the at least one first data object includes medical claims data and the at least one second data object includes medical note data.
Example 15. A system for fall event detection, the system comprising: at least one memory having processor-readable instructions stored therein; and at least one processor configured to access the at least one memory and execute the processor-readable instructions to perform operations, the operations comprising: receiving at least one first data object for an entity; receiving at least one second data object for the entity, the at least one second data object being associated with the at least one first data object; determining if the at least one first data object includes at least one inclusionary code associated with a physical injury, the at least one inclusionary code being indicative of a fall-related injury; determining if the at least one first data object excludes at least one exclusionary code associated with a physical injury, the at least one exclusionary code being indicative of a non-fall-related injury; and when it is determined that the at least one first data object includes the at least one inclusionary code and excludes the at least one exclusionary code: marking the at least one first data object as being indicative of a possible fall event; determining if the at least one second data object includes at least one fall-related word; and when it is determined that the at least one second data object includes the at least one fall-related word, marking the at least one first data object as being indicative of an actual fall event.
Example 16. The system of Example 15, wherein determining if the at least one second data object includes the at least one fall-related word includes performing a lexical search for the at least one fall-related word in the at least one second data object and satisfying a contextual match.
Example 17. The system of any of Examples 15-16, wherein determining if the at least one first data object includes the at least one inclusionary code and excludes the at least one exclusionary code includes performing a search of one or more individual claim lines of the at least one first data object to detect a presence of the at least one inclusionary code and detect an absence of the at least one exclusionary code.
Example 18. The system of any of Examples 15-17, wherein when it is determined that the at least one second data object includes the at least one fall-related word, transmitting a notification to a computing device associated with a healthcare provider that the entity has likely experienced the actual fall event.
Example 19. The system of any of Examples 15-18, wherein when it is determined that the at least one second data object includes the at least one fall-related word, notifying the entity of an available medical intervention via a first computing device associated with the entity.
Example 20. A non-transitory computer-readable medium storing a set of instructions that, when executed by at least one processor, cause the at least one processor to perform operations comprising: receiving at least one first data object for an entity; receiving at least one second data object for the entity, the at least one second data object being associated with the at least one first data object; determining if the at least one first data object includes at least one inclusionary code associated with a physical injury, the at least one inclusionary code being indicative of a fall-related injury; determining if the at least one first data object excludes at least one exclusionary code associated with a physical injury, the at least one exclusionary code being indicative of a non-fall-related injury; and when it is determined that the at least one first data object includes the at least one inclusionary code and excludes the at least one exclusionary code: marking the at least one first data object as being indicative of a possible fall event; determining if the at least one second data object includes at least one fall-related word; and when it is determined that the at least one second data object includes the at least one fall-related word, marking the at least one first data object as being indicative of an actual fall event.

What is claimed is:

1. A method performed by one or more processors of a computing system, the method comprising:
receiving at least one first data object for an entity;
receiving at least one second data object for the entity, the at least one second data object being associated with the at least one first data object;
determining if the at least one first data object includes at least one inclusionary code associated with a physical injury, the at least one inclusionary code being indicative of a fall-related injury;
determining if the at least one first data object excludes at least one exclusionary code associated with a physical injury, the at least one exclusionary code being indicative of a non-fall-related injury; and
when it is determined that the at least one first data object includes the at least one inclusionary code and excludes the at least one exclusionary code:
marking the at least one first data object as being indicative of a possible fall event;
determining if the at least one second data object includes at least one fall-related word; and
when it is determined that the at least one second data object includes the at least one fall-related word, marking the at least one first data object as being indicative of an actual fall event.

2. The method of claim 1, wherein the at least one inclusionary code and the at least one exclusionary code each include a diagnosis code.

3. The method of claim 1, wherein the at least one inclusionary code includes a W-series ICD code or a S-series ICD code.

4. The method of claim 1, wherein the at least one inclusionary code includes a R29.6 ICD-10 code.

5. The method of claim 1, wherein the at least one exclusionary code includes a V-series ICD code, a W-series ICD code, an X-series ICD code, or a Y-series ICD code.

6. The method of claim 1, wherein determining if the at least one second data object includes the at least one fall-related word includes performing a lexical search for the at least one fall-related word in the at least one second data object and satisfying a contextual match.

7. The method of claim 1, wherein the at least one inclusionary code includes a diagnostic code associated with one or more diagnostic positions associated with the entity.

8. The method of claim 1, wherein the at least one first data object includes historical medical claims data for the entity, the historical medical claims data spanning a duration of three or more years, the historical medical claims data including:
one or more historical fall events associated with one or more historical physical injuries, the one or more historical fall events including the actual fall event, the one or more historical fall events being identified based on inclusion of the at least one inclusionary code, exclusion of the at least one exclusionary code, and inclusion of the at least one fall-related word in the at least one second data object.

9. The method of claim 8, further comprising:
receiving informational data associated with the entity, the informational data including at least one of demographics data, diagnosis data, or medication data,
wherein the demographics data includes data related to age, gender, income, or region of residence associated with the entity,
wherein the diagnosis data includes data related to current or previous diagnoses associated with the entity, and
wherein the medication data includes data related to current or previous medications prescribed to the entity;
training a machine-learning model based on the one or more historical fall events and the informational data, the machine-learning model being configured to determine a relationship between the one or more historical fall events and the informational data; and
determining if a different entity is likely to have a future fall event based on the machine-learning model.

10. The method of claim 1, wherein determining if the at least one first data object includes the at least one inclusionary code and excludes the at least one exclusionary code includes performing a search of one or more individual claim lines of the at least one first data object to detect a presence of the at least one inclusionary code and detect an absence of the at least one exclusionary code.

11. The method of claim 1, wherein when it is determined that the at least one second data object includes the at least one fall-related word, notifying the entity of an available medical intervention via a first computing device associated with the entity.

12. The method of claim 1, wherein when it is determined that the at least one second data object includes the at least one fall-related word, modifying a third data object stored in a data store to include that the entity is associated with the actual fall event.

13. The method of claim 1, wherein when it is determined that the at least one second data object includes the at least one fall-related word, transmitting a notification to a computing device associated with a healthcare provider that the entity has likely experienced the actual fall event.

14. The method of claim 1, wherein the at least one first data object includes medical claims data and the at least one second data object includes medical note data.

15. A system for fall event detection, the system comprising:
at least one memory having processor-readable instructions stored therein; and
at least one processor configured to access the at least one memory and execute the processor-readable instructions to perform operations, the operations comprising:
receiving at least one first data object for an entity;
receiving at least one second data object for the entity, the at least one second data object being associated with the at least one first data object;
determining if the at least one first data object includes at least one inclusionary code associated with a physical injury, the at least one inclusionary code being indicative of a fall-related injury;
determining if the at least one first data object excludes at least one exclusionary code associated with a physical injury, the at least one exclusionary code being indicative of a non-fall-related injury; and
when it is determined that the at least one first data object includes the at least one inclusionary code and excludes the at least one exclusionary code:
marking the at least one first data object as being indicative of a possible fall event;
determining if the at least one second data object includes at least one fall-related word; and
when it is determined that the at least one second data object includes the at least one fall-related word, marking the at least one first data object as being indicative of an actual fall event.

16. The system of claim 15, wherein determining if the at least one second data object includes the at least one fall-related word includes performing a lexical search for the at least one fall-related word in the at least one second data object and satisfying a contextual match.

17. The system of claim 15, wherein determining if the at least one first data object includes the at least one inclusionary code and excludes the at least one exclusionary code includes performing a search of one or more individual claim lines of the at least one first data object to detect a presence of the at least one inclusionary code and detect an absence of the at least one exclusionary code.

18. The system of claim 15, wherein when it is determined that the at least one second data object includes the at least one fall-related word, transmitting a notification to a computing device associated with a healthcare provider that the entity has likely experienced the actual fall event.

19. The system of claim 15, wherein when it is determined that the at least one second data object includes the at least one fall-related word, notifying the entity of an available medical intervention via a first computing device associated with the entity.

20. A non-transitory computer-readable medium storing a set of instructions that, when executed by at least one processor, cause the at least one processor to perform operations comprising:
receiving at least one first data object for an entity;
receiving at least one second data object for the entity, the at least one second data object being associated with the at least one first data object;
determining if the at least one first data object includes at least one inclusionary code associated with a physical injury, the at least one inclusionary code being indicative of a fall-related injury;
determining if the at least one first data object excludes at least one exclusionary code associated with a physical injury, the at least one exclusionary code being indicative of a non-fall-related injury; and
when it is determined that the at least one first data object includes the at least one inclusionary code and excludes the at least one exclusionary code:
marking the at least one first data object as being indicative of a possible fall event;
determining if the at least one second data object includes at least one fall-related word; and
when it is determined that the at least one second data object includes the at least one fall-related word, marking the at least one first data object as being indicative of an actual fall event.
